(19) United States
(12) Patent Application Publication
(10) Pub. No.: US 2015/0284685 A1
(43) Pub. Date: Oct. 8, 2015

(54) DEVICE AND METHOD FOR OPENING EXTRACTED TEETH AND REMOVAL OF PULP

(71) Applicant: Dharminder Happy Singh Ghag, Las Vegas, NV (US)

(72) Inventor: Dharminder Happy Singh Ghag, Las Vegas, NV (US)

(21) Appl. No.: 14/244,905

(22) Filed: Apr. 4, 2014

(51) Int. Cl. C12N 5/0775

(52) U.S. Cl. CPC C12N 5/0664

(57) ABSTRACT

A tooth cracking device improves the efficiency of removal of pulp from extracted teeth. The device may have:
a frame having a central open area; the frame having two opposed surfaces within the open area;
on a first of the two opposed surfaces is an at least three-prong support;
on a second of the two opposed surfaces, and opposed to the at least three-prong support is a tapered penetrating probe;
   wherein the tapered penetrating probe is controllably extendable from the second of the two opposed surfaces.

### BACKGROUND OF THE INVENTION

1. Field of the Invention

The present invention relates to the field of tooth extraction and the harvesting of pulp and stem cells from the extracted teeth by opening the extracted tooth and removing the pulp.

2. Background of the Art

Dental Pulp Stem Cells, or (DPSCs) are multipotent stem cells that have the potential to differentiate into a variety of cell types. More recently a subpopulation of dental pulp stem cells has been described as human Immature Dental Pulp Stem Cells (IDPSC). There are various studies where the importance of these cells and their regenerative capacity has been demonstrated. Through the addition of tissue-specific cytokines, differentiated cells were obtained in vitro from these cells, not only of mesenchymal linage but also of endo- and ecto-dermal linage. Among them are the IPS, MAPCs cells.

Several publications have stressed the importance of the expression of pluripotentiality associated markers: the transcription factors Nanog, Sox2, Oct3/4, SSEA4, CD13, are indispensable for the stem cells to divide indefinitely without affecting their differentiation potential, i.e., maintaining their self-renovation capacity. The quantification of protein expression levels in these cells is very important in order to know their pluripotentiality level, as described in some publications.

A protocol for isolating and identifying the subpopulations of pluripotent- like stem cells from the dental pulp (DPSC) has been established (Atari M, Gil-Recio C, Fabregat M, García-Fernández D A, Barajas M, Carrasco M, Jung H S, Hernandez-Alfaro F, Casals N, Prosper F, Ferrés Padro E, Giner L. Dental Pulp of the Third Molar: A New Source of Pluripotent-like Stem Cells.J Cell Sci. 2012 Mar. 30.) These cells are SSEA4+, OCT3/4+, NANOG+, SOX2+, LIN28+, CD13+, CD105+, CD34−, CD45−, CD90+, CD29+, CD73+, STRO1+and CD146−, and they show genetic stability in vitro based on genomic analysis with a newly described CGH technique.

DPSCs were able to form both embryoid bodies-like structures (EBs) in vitro and teratoma-like structures that contained tissues derived from all three embryonic germ layers when injected in nude mice. DPSCs can differentiate in vitro into tissues that have similar characteristics to mesoderm, endoderm and ectoderm layers.

Dental pulp is the soft living tissue inside a tooth. Stem Cells are found inside the soft living tissue. Scientists have identified the mesenchymal type of stem cell inside dental pulp. This particular type of stem cell has the future potential to differentiate into a variety of other cell types including: Myocardiocytes to repair damaged cardiac tissue following a heart attack;

Neuronal to generate nerve and brain tissue; Myocytes to repair muscle; Osteocytes to generate bone; Chondocytes to generate cartilage; Adipocytes to generate fat; and Bone and tissue from the oral cavity.

It has also become medically desirable to preserve stem cells for individuals, especially children for potential use in later techniques where stem cells might be essential starting components.

The present methods for removing pulp from teeth (after extraction) are fairly primitive, which is surprising in view of the value of the material. Extracted teeth are cracked with any available instrument such as tongs, pliers and the like, with exposed pulp scraped out, along with fragmented bone. Wires may be used for the scraping. Holes may be drilled into the extracted tooth, and wire inserted into the hole to pull out the pulp. These methods are crude, inefficient and can damage significant portions of the cells.

### SUMMARY OF THE INVENTION

A process for cracking teeth and removing pulp uses a tooth cracking device having:

a frame with a central open area; the frame having two opposed surfaces within the open area; on a first of the two opposed surfaces is an at least three-prong support; on a second of the two opposed surfaces, and opposed to the at least three-prong support is a tapered penetrating probe. The tapered penetrating probe is controllably extendable from the second of the two opposed surfaces to breach and split a tooth restrained in the three-prong support.

### BRIEF DESCRIPTION OF THE FIGURES

FIG. 1 shows a partially exploded side view of a tooth cracking device according to the present technology.

### DETAILED DESCRIPTION OF THE INVENTION

A tooth cracking device improves the efficiency of removal of pulp from extracted teeth. The device may have:
a frame having a central open area;
the frame having two opposed surfaces within the open area;
on a first of the two opposed surfaces is an at least three-prong support;
on a second of the two opposed surfaces, and opposed to the at least three-prong support is a tapered penetrating probe;
   wherein the tapered penetrating probe is controllably extendable from the second of the two opposed surfaces.

An alternative description of the tooth-cracking device is as a tooth cracking device has at least: a frame comprising a central open area and two pairs of opposed arms defining the open area; the frame having two opposed surfaces on one pair of arms forming the open area; associated with a first of the two opposed surfaces is an at least three-prong support; and associated with a second of the two opposed surfaces, and opposed to the at least three-prong support is a tapered penetrating probe. The tapered penetrating probe may be controllably extendable from the second of the two opposed surfaces. The tooth cracking device may have one or both of the three-prong support and/or the tapered penetrating tube pass through an opening in the first or second of the two opposed surfaces, respectively.

The frame should be relatively rigid (e.g.; sturdy metal frame, polymeric frame, composite frame and the like) so that forces generated during the forced splitting or cracking of the tooth does not bend the frame sufficiently to alter the trajectory and/or alignment of the tapered penetrating probe. The tapered penetrating probe may advance by screw action or stepped advances (clicks, or snaps, for example) from the second of the two opposed surfaces. The at least three prong support may be on a base support (flat on the bottom, flat or rounded on the top) that is secured or locked to the second of the two opposed surfaces. The base may be locked through a locking system selected from the group consisting of snaps, screws, or cams, which may be unlocked to enable removal of the prong support for replacement or cleaning.

The prongs may have the appearance of gem supports on jewelry. The prongs may radiate from the base support, initially extending away from each other to create a widening support area, and then bending inwardly to provide a grasping function at tips of the prongs of the support. The at least three-prong support may be at least four prongs. The tapered penetrating probe has a conical taper (the cross-section may be any shape, such as circular, oval, square, rectangular, serrated, and/or vary in shape) that may be configured to advance towards and between the at least three prongs of the at least three prong support, where the extracted tooth is supported. The frame may be formed from two distinct opposed segments which can be temporarily secured together, as by posts passing from one frame to another into receptor holes. External screws on the frames may penetrate sides of the frames and lock posts into the frames and thereby securing the frame together. The tooth cracking device may have the frame as two distinct opposed segments which can be temporarily secured together and the tapered penetrating probe has a conical taper that is configured to advance towards and between the at least three prongs of the at least three prong support.

A method of extracting pulp from an extracted tooth comprising placing the extracted tooth between the at least three prongs of the tooth cracking device described above by stabilizing the tooth within the prongs against free movement, advancing the tapered penetrating probe into the extracted tooth while the tooth is supported between the at least three prongs, extending the probe into the tooth to cause the tooth to split outwardly from the extended probe while exterior surfaces of the tooth are supported by the at least three prongs, removing the extended probe from the split tooth, exposing pulp from inside the split tooth by separating pieces of the split tooth, and removing the pulp from the separated pieces of the split tooth.

The sides of the tooth (and optionally the crown of the tooth) is preferably score (a groove placed on the surface) to assist in defining lines along which the tooth will be more easily split by the device. The scoring may be done by hand before or after the tooth is supported by the prongs. Preferably at least two, three or four score lines are cut into the sides of the tooth.

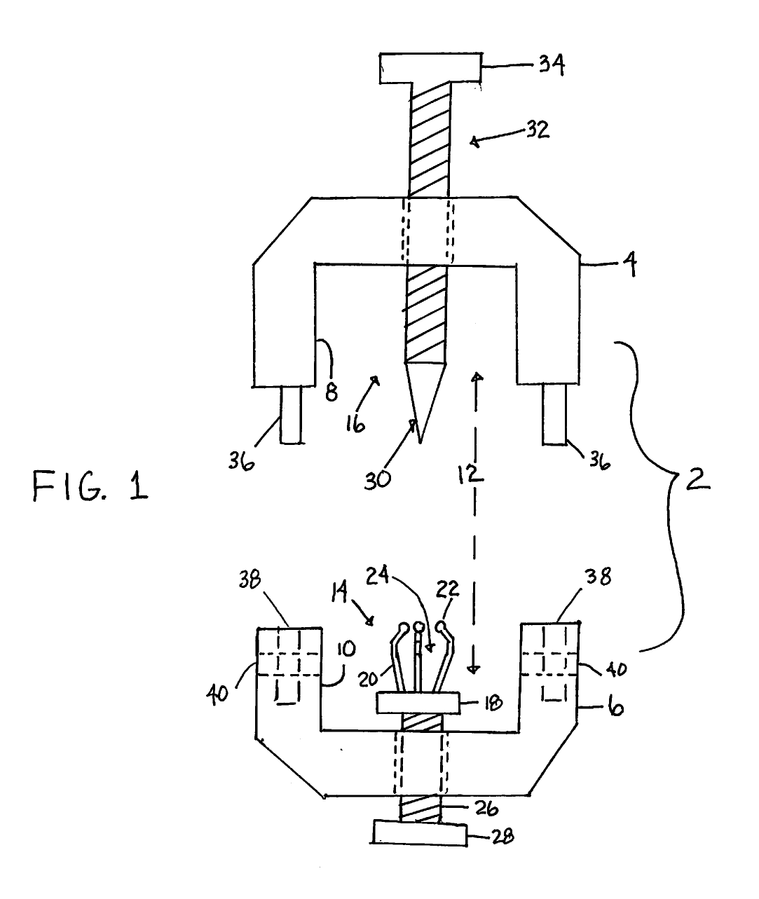

Reference to the FIGURE will assist in a better appreciation of the present technology. FIG. 1 shows a partially exploded side view of a tooth cracking device 2 according to the present technology. The device 2 is shown with a top half frame 4 and a bottom half frame 6. The inside opposed surface 8 of the top frame 4 and the second opposed inside surface 10 of the bottom frame 6 are shown. When the top half frame 4 and the bottom half frame 6 are moved together along line 12, the two half frames 4 and 6 close to form an open area including the line 12. Within that open area would be included the at least three-prong support device 14 and the opposed extendable tapered penetrating device 16.

The at least three-prong support component 14 is shown with three prongs 20, each prong shown with a preferable support tip 22. The three prongs 20 create a capture area 24, within which a tooth (not shown) would be securely stabilized during cracking, and the prongs 20 also support a tooth after cracking to prevent loss of larger tooth segments. The at least three prongs 20 are shown supported on a base 18 which an be adjusted with respect to the half frame 6 by threaded advancement using threads 26 and turning the base 18 using finger grip 28. The base 18 may swivel freely off the end of the threads 26 so that advancing the base 18 does not rotate the base 18 or its supported at least three prongs 20.

The opposed extendable tapered penetrating device 16 has a penetrating tip 30 which may be conial (as previously described), but may also be a blade, or other sharpened or tapering end. The tapering is to assist in increasing expanding forces from within the tooth as the penetration of the tip 30 into the tooth increases. A non-tapered element can be used, but that would tend to initially create larger crushed enamel particles before the tooth is split. Such a tip is therefore less preferred. The opposed extendable tapered penetrating device 16 is also shown with a thread advancing surface 32 and a separate finger grip 34. The tip 30 may or may not swivel. The top frame 4 is also shown with a pair of male posts 36 that, when the top half frame 4 is advanced to engage the bottom half frame 6, the posts enter female receptor openings 38 to align and partially secure the two half frames 4 and 6 together. The two half frames 4 and 6 may be further secured by providing threaded tightening posts (not shown) into perpendicular locking holes 40 to tighten against the male posts 36 when inserted into the female receptors 38.

In operating the system 2, the two half frames 4 and 6 would be engaged, before or after the tooth was secured within the at least three prong support 14. The opposed extendable tapered penetrating device 16 would be advanced towards the (preferably scored) supported tooth. Once contact was made with the tooth by the tip 30, the thumb advancing grip 34 would continue to be turned, the tip 30 advancing further into the tooth (not shown), increasing sideways, internal pressure is applied against the tooth, and the forces from the pressure then split/fracture/crack the tooth while it is supported by the at least three prongs 20 and further restrained vertically by the tips 22. After the tooth has been split, the pieces of the tooth may be removed in a number of different ways. The base 18, prongs 20 and the supported tooth may be disengaged from the threaded advancing element 26, or the element 26 may be removed from the finger grip 28 and the base 18 removed from the device 2.

Alternatively, the two half frames 4 and 6 may be separated from each other and the bottom half frame 6 used as an operation stage during removal of pulp from the split tooth. Still alternatively, after separation of the two half frames 4 and 6, the segments of the split/cracked tooth may be removed from the at least three prongs 20 and the pulp then removed. Because the degree of splitting of the tooth can be better controlled using the present device and process, and more efficient retrieval of pulp can be provided. In addition, by accurately scoring the outside of the tooth before cracking, more exact locations of break lines can be provided, more uniform tooth segments can be provided, and more of the pulp can be exposed by the initial cracking so that excessive excavation or further manual tool breaking of the segments would not be necessary. The scoring of the tooth may be performed with any convenient instrumentality, such as a carbide wheel, diamond wheel, a blade, a laser, rotatory instrument, or a hand instrument. Scoring need only be deep enough to create a line of less resistance along which a crack or a split may propagate.

What is claimed:

1. A tooth cracking device comprising:
a frame having a central open area;
the frame having two opposed surfaces within the open area;
on a first of the two opposed surfaces is an at least three-prong support;
on a second of the two opposed surfaces, and opposed to the at least three-prong support is a tapered penetrating probe;
wherein the tapered penetrating probe is controllably extendable from the second of the two opposed surfaces.

2. The tooth cracking device of claim 1 wherein the tapered penetrating probe advances by screw action from the second of the two opposed surfaces.

3. The tooth cracking device of claim 1 wherein the at least three prong support is on a base support that is locked to the second of the two opposed surfaces.

4. The tooth cracking device of claim 3 wherein the base is locked through a locking system selected from the group consisting of snaps, screws, or cams.

5. The tooth cracking device of claim 1 wherein the at least three-prong support comprises four prongs.

6. The tooth cracking device of claim 1 wherein the tapered penetrating probe has a conical taper that is configured to advance towards and between the at least three prongs of the at least three prong support.

7. The tooth cracking device of claim 2 wherein the tapered penetrating probe has a conical taper that is configured to advance towards and between the at ;least three prongs of the at least three prong support.

8. The tooth cracking device of claim 4 wherein the tapered penetrating probe has a conical taper that is configured to advance towards and between the at ;least three prongs of the at least three prong support.

9. The tooth cracking device of claim 2 wherein the frame comprises two distinct opposed segments which can be temporarily secured together.

10. The tooth cracking device of claim 5 wherein the frame comprises two distinct opposed segments which can be temporarily secured together.

11. The tooth cracking device of claim 10 wherein the frame comprises two distinct opposed segments which can be temporarily secured together and the tapered penetrating probe has a conical taper that is configured to advance towards and between the at least three prongs of the at least three prong support.

12. A method of extracting pulp from an extracted tooth comprising placing the extracted tooth between the at least three prongs of the tooth cracking device of claim 1, stabilizing the tooth against free movement, advancing the tapered penetrating probe into the extracted tooth while the tooth is supported between the at least three prongs, extending the probe into the tooth to cause the tooth to split outwardly from the extended probe while exterior surfaces of the tooth are supported by the at least three prongs, removing the extended probe from the split tooth, exposing pulp from inside the split tooth by separating pieces of the split tooth, and removing the pulp from the separated pieces of the split tooth.

13. A method of extracting pulp from an extracted tooth comprising placing the extracted tooth between the at least three prongs of the tooth cracking device of claim 2, stabilizing the tooth against free movement, advancing the tapered penetrating probe into the extracted tooth while the tooth is supported between the at least three prongs, extending the probe into the tooth to cause the tooth to split outwardly from the extended probe while exterior surfaces of the tooth are supported by the at least three prongs, removing the extended probe from the split tooth, exposing pulp from inside the split tooth by separating pieces of the split tooth, and removing the pulp from the separated pieces of the split tooth.

14. A method of extracting pulp from an extracted tooth comprising placing the extracted tooth between the at least three prongs of the tooth cracking device of claim 4, stabilizing the tooth against free movement, advancing the tapered penetrating probe into the extracted tooth while the tooth is supported between the at least three prongs, extending the probe into the tooth to cause the tooth to split outwardly from the extended probe while exterior surfaces of the tooth are supported by the at least three prongs, removing the extended probe from the split tooth, exposing pulp from inside the split tooth by separating pieces of the split tooth, and removing the pulp from the separated pieces of the split tooth.

15. A method of extracting pulp from an extracted tooth comprising placing the extracted tooth between the at least three prongs of the tooth cracking device of claim 5, stabilizing the tooth against free movement, advancing the tapered penetrating probe into the extracted tooth while the tooth is supported between the at least three prongs, extending the probe into the tooth to cause the tooth to split outwardly from the extended probe while exterior surfaces of the tooth are supported by the at least three prongs, removing the extended probe from the split tooth, exposing pulp from inside the split tooth by separating pieces of the split tooth, and removing the pulp from the separated pieces of the split tooth.

16. A method of extracting pulp from an extracted tooth comprising placing the extracted tooth between the at least three prongs of the tooth cracking device of claim 8, stabilizing the tooth against free movement, advancing the tapered penetrating probe into the extracted tooth while the tooth is supported between the at least three prongs, extending the probe into the tooth to cause the tooth to split outwardly from the extended probe while exterior surfaces of the tooth are supported by the at least three prongs, removing the extended probe from the split tooth, exposing pulp from inside the split tooth by separating pieces of the split tooth, and removing the pulp from the separated pieces of the split tooth.

17. The method of claim 12 wherein the tooth is scored along its outside surface to assist in defining break lines for the tooth.

18. The method of claim 13 wherein the tooth is scored along its outside surface to assist in defining break lines for the tooth.

19. The method of claim 14 wherein the tooth is scored along its outside surface to assist in defining break lines for the tooth.

20. A tooth cracking device comprising:
a frame comprising a central open area and two pairs of opposed arms defining the open area;
the frame having two opposed surfaces on one pair of arms forming the open area;
associated with a first of the two opposed surfaces is an at least three-prong support; and
associated with a second of the two opposed surfaces, and opposed to the at least three-prong support is a tapered penetrating probe;
wherein the tapered penetrating probe is controllably extendable from the second of the two opposed surfaces.

21. The tooth cracking device of claim 20 wherein the tapered penetrating tube passes through an opening in the second of the two opposed surfaces.
